(19) United States
(12) Patent Application Publication
(10) Pub. No.: US 2014/0238401 A1
(43) Pub. Date: Aug. 28, 2014

(54) CPAP SYSTEM

(71) Applicant: Walter Jent Paschall, Ball Ground, GA (US)

(72) Inventor: Walter Jent Paschall, Ball Ground, GA (US)

(21) Appl. No.: 14/187,208

(22) Filed: Feb. 21, 2014

Related U.S. Application Data
(60) Provisional application No. 61/768,353, filed on Feb. 22, 2013.

(51) Int. Cl. A61M 16/06; A61M 16/00; A61M 16/08

(52) U.S. Cl. CPC A61M 16/0622; A61M 16/0683; A61M 16/0816; A61M 16/0875; A61M 16/0066
USPC 128/205.25

(57) ABSTRACT

The technology described herein provides an apparatus and associated methods for Continuous Positive Airway Pressure (CPAP) systems, which can be utilized to treat breathing problems such as sleep apnea. In at least one embodiment, an airway interface mask and headgear assembly includes a mask shell housing to cover the patients nose and mouth and a pillowed, non-collapsible air cushion consisting of a continuously sealed, generally circular, and hollow cylinder which is limited in compression and which is disposed upon the mask shell housing sealingly in a generally circular manner on an underside of the mask housing to engage the patient's face. The pillowed, non-collapsible air cushion provides the sealed, cushioned engagement to the patient's face necessary, with conformity to the shape and contours of the patient's face, in the CPAP system without allowing for compression of the mask shell housing to allow the mask housing to touch the patient's face.

### CROSS-REFERENCE TO RELATED APPLICATION(S)

The present non-provisional patent application claims the benefit of priority of U.S. Provisional Patent Application No. 61/768,353, which is entitled “CPAP SYSTEM”, which was filed on Feb. 22, 2014, and which is incorporated in full by reference herein.

### FIELD OF THE INVENTION

The technology described herein relates generally the fields of medical devices and treatment systems. More specifically, this technology relates to Continuous Positive Airway Pressure (CPAP) systems, which can be utilized to treat breathing problems such as sleep apnea, and the like.

### BACKGROUND OF THE INVENTION

Continuous Positive Airway Pressure (CPAP) systems provide treatment utilizing mild air pressure to keep a user's airways open. CPAP systems are typically used to aid persons with breathing problems such as sleep apnea. However, CPAP systems can be utilized in other manners, such as for the treatment of preterm infants whose lungs have not fully developed.

Sleep apnea is a sleep and breathing disorder characterized by abnormal pauses in breathing or instances of abnormally low breathing. Each pause in breath is called an apnea. Each instance of low breathing is called a hyponea. Sleep apnea can occur is several forms: central, obstructive, and complex. In central sleep apnea, breathing is interrupted by a lack of respiratory effort. In obstructive sleep apnea, breathing is interrupted by a physical block to airflow despite respiratory effort, and snoring is common. Complex sleep apnea is a combination of central and obstructive sleep apnea.

CPAP devices are known to be successful in the treatment of sleep apnea patients, particularly obstructive sleep apnea. Typically, a CPAP system includes: 1) a mask or other device that fits over your nose or your nose and mouth, with straps to keep the mask in place while worn; 2) a tube that connects the mask to the motor of the CPAP system; and 3) a motor that blows air into the tube. Optional components, such as humidifiers or heated humidifiers, filters, air purifiers, and so forth, can be utilized with a CPAP system.

Related utility patents known in the art include the following:

U.S. Pat. No. 6,581,594, issued to Drew et al. on Jun. 24, 2003, discloses a vent assembly suitable for use with a respiratory mask of the type used in CPAP treatment. In one embodiment the vent is made of a thin air permeable membrane. Generally, the membrane is thinner than 0.5 mm. The membrane can be made of a hydrophobic material such as polytetrafluoroethylene (PTFE). The membrane can also be fabricated from expanded PTFE. The expanded PTFE membrane is mounted on a polypropylene scrim. The pores of the membrane have a reference pore size of 10 to 15 microns. In an alternative embodiment, the vent assembly includes a vent constructed from stainless steel. In another embodiment the membrane has a superficial cross-sectional area of approximately 500 mm². In another embodiment the vent assembly comprises a membrane attached to a vent frame, the vent assembly forming an insert which can be removeably attached to a mask frame.

U.S. Pat. No. 6,691,707, issued to Gunaratnam et al. on Feb. 17, 2004, discloses a connector for a respiratory make and a respiratory mask. A connector includes a mask end for connecting with the interior of a respiratory mask, a supply conduit end, and a gas washout vent passage having an inlet adjacent to, or forming part of, the mask end in fluid communication with the interior of the respiratory mask and an outlet in fluid communication with atmosphere. The vent outlet of the vent passage is disposed on the side of the connector remote from the mask end. A respiratory mask includes a mask shell, a mask inlet for connecting with the outlet of a breathable gas supply conduit, and a gas washout vent passage. A vent outlet is disposed on the side of the mask remote from the mask interior and is adapted to direct the washout gas in a direction substantially parallel to the longitudinal axis of the mask inlet and away from the mask inlet.

U.S. Pat. No. 6,981,503, issued to Shapiro on Jan. 3, 2006, discloses a hands-fee anesthesia mask. A hands-fee anesthesia mask includes a mask for providing anesthesia to the patient, a mask strap for holding the mask against a patient's mouth, and a jaw raising strap for raising the patient's jaw up and forward so as to unobstruct the patient's airway.

U.S. Pat. No. 7,188,620, issued to Amarasinghe on Mar. 13, 2007, discloses a headgear. A headgear for securing and positioning a mask suitable for the treatment of sleep disordered breathing is constructed from a composite including polyurethane foam. It includes a back portion with upper and lower straps connected to the back portion. The straps have relatively narrow strap ends the lower straps are displaced downward with respect to the back portion. A quick release mechanism near the front of the face attaches the headgear to the mask. Additional components can be attached to the straps to alter their elasticity and stiffness.

U.S. Pat. No. 7,472,703, issued to Hernandez et al. on Jan. 6, 2009, discloses a ventilation comfort interface. A soft material replaceably coupled to a ventilation interface, wherein the soft material is positioned between the interface and a user's face to alleviate discomfort. The soft material may be configured to be replaceably couple to various ventilation interfaces which may include CPAP interfaces.

U.S. Pat. No. 7,845,354, issued to Kwok et al. on Dec. 7, 2010, discloses a mask and vent assembly. A mask is disclosed for use with a system for supplying breathable gas pressurized above atmospheric pressure to a human or animal's airways. The mask includes a mask shell which is, in use, in fluid communication with a gas supply conduit and a gas washout vent assembly. The gas washout vent assembly includes at least one gas washout orifice extending from a first side of the vent assembly positioned, in use, adjacent the human or animal's face and a second side positioned, in use, adjacent the atmosphere. The cross-sectional area of the orifice at the first side is larger than the cross-sectional area of the orifice at the second side.

U.S. Pat. No. 8,042,541, issued to Amarasinghe et al. on Oct. 25, 2011, discloses a mask brace and mask assembly. A patient mask assembly for positive pressure ventilation includes a brace which clips to the mask and provides attachment locations for headgear straps in alternative positions to those attachment locations which may be provided by the mask. The brace may also provide a mask stabilizing portion.

Related published utility patent applications known in the art include the following:

U.S. Patent Application Publication No. 2004/0094159, filed by Kwok et al. and published on May 20, 2004, discloses a breathing mask and mask cushion. A full-face cushion comprises a substantially triangularly shaped frame from which extends a membrane. The frame has a scalloped edge by which the cushion is affixed to a mask body to form a full-face mask. The membrane has an aperture into which the wearer's nose is received. The member is spaced away from the rim of the frame, and its outer surface is of substantially the same shape as the rim. Respective notches receive the bridge of the wearer's nose. The wearer's nose and lips are received through the aperture into the chamber within the mask body. The seal forming portion thus contacts both the surface of the wearer's nose and a portion of the wearer's face in the region between the bottom lip and the chin, and around the sides and over the bridge of the nose. The shape of the seal forming portion is particularly suited to effectively seal the difficult region of the facial contour that is the crease between the sides of the nose and the face.

U.S. Patent Application Publication No. 2009/0139525, filed by Schirm and published on Jun. 4, 2009, discloses a comfort liner for pressure face masks and nasal masks. Disclosed is a device and method for improving the facial comfort and sealing ability of a full-face mask. The device comprises a liner of soft material, preferably made from moleskin or the like. The disclosed face mask liner provides a superior feel to the user's face compared with standard face masks with air-filled cushion or silicone gasket cuffs, while providing a more comfortable user experience and a superior seal to prevent loss of breathing gas. Also disclosed is a similar device and method for improving the facial comfort of a nasal mask, providing a mask that is more comfortable to the user.

Related design patent applications known in the art include the following:

U.S. Pat. No. D644,729, issued to Ferris et al. on Sep. 6, 2011, discloses the ornamental design for a padded mask for use with CPAP.

The foregoing patent and other information reflect the state of the art of which the inventor is aware and are tendered with a view toward discharging the inventor's acknowledged duty of candor in disclosing information that may be pertinent to the patentability of the technology described herein. It is respectfully stipulated, however, that the foregoing patent and other information do not teach or render obvious, singly or when considered in combination, the inventor's claimed invention.

### BRIEF SUMMARY OF THE INVENTION

In various exemplary embodiments, the technology described herein provides an apparatus and associated methods for a Continuous Positive Airway Pressure (CPAP) system, which can be utilized to treat breathing problems such as sleep apnea, and the like. Continuous Positive Airway Pressure (CPAP) systems provide treatment utilizing mild air pressure to keep a user's airways open. CPAP systems are typically used to aid persons with breathing problems such as sleep apnea.

In one exemplary embodiment, the technology described herein provides an airway interface mask and headgear assembly for a patient Continuous Positive Airway Pressure (CPAP) system, utilized to treat breathing problems such as sleep apnea utilizing mild air pressure to keep a user's airways open. The airway interface mask and headgear assembly includes: a mask shell housing to cover the patient's at least one airway interface, such as a mouth, nose, or both; and a pillowed, non-collapsible air cushion consisting of a continuously sealed, generally circular, and hollow cylinder which is limited in compression and which is disposed upon the mask shell housing sealingly in a generally circular manner on an underside of the mask housing to engage the patient's face. The pillowed, non-collapsible air cushion provides the sealed, cushioned engagement to the patient's face necessary, with conformity to the shape and contours of the patient's face, in the CPAP system without allowing for compression of the mask shell housing to allow the mask housing to touch the patient's face.

In at least one embodiment of the airway interface mask and headgear assembly, the assembly also includes an outer ring, disposed around a perimeter of the mask shell housing most proximate to the patient's face when worn, to provide structural support the mask shell housing and with which to couple to the pillowed, non-collapsible air cushion.

In at least one embodiment of the airway interface mask and headgear assembly, the assembly further includes an adapter ring, disposed around a perimeter of the mask shell housing most distal to the patient's face when worn, to provide structural support the mask shell housing and further comprising a fluid inlet/outlet for air passage with which to couple to an air hose of a CPAP system and provide airflow to the patient through the mask shell housing to the CPAP system.

In at least one embodiment of the airway interface mask and headgear assembly, the assembly also includes: a plurality of headgear connection points; and a protrusion ring, disposed around a perimeter of the adapter ring, to provide the plurality of headgear connection points.

In at least one embodiment of the airway interface mask and headgear assembly, the assembly further includes: a harness with which to couple the airway interface mask to the patient's face; a plurality of straps disposed about the harness; a plurality of apertures disposed about each of the plurality of straps to provide a plurality of connectivity points to the patient to provide multi-point adjustability and to adjust for tightness and comfort; and a plurality of reinforcement lips disposed about at least one side of the plurality of straps at the circumference of each of the plurality of apertures.

In at least one embodiment of the airway interface mask and headgear assembly, the assembly also includes a swivel coupled to the adapter ring to allow an air hose to rotate without twisting and without placing pressure on the mask shell housing while being worn by the patient.

In at least one embodiment of the airway interface mask and headgear assembly, the assembly further includes an air insulation cuff comprised of non-porous foam, disposed upon the pillowed, non-collapsible air cushion on a side most proximate to placement upon the patient's face, to provide additional comfort and conformity to the shape and contours of the patient's face.

In at least one embodiment of the airway interface mask and headgear assembly, the assembly also includes: a headgear frame, with which to couple the airway interface mask to the patient's head, the headgear frame comprising a first band for placement about the top portion of the patient's head, from first ear to back of head, to second ear, to forehead, and back to the first ear; and a first tension adjustment knob disposed upon the first band at the back of the head position to tighten or loosen the tension within the headgear frame along the first band.

In at least one embodiment of the airway interface mask and headgear assembly, the assembly further includes: a second band disposed upon the headgear frame for placement about the top portion of the patient's head, from first ear to second ear over the top most portion of the head; and a second tension adjustment knob disposed upon the second band to tighten or loosen the tension within the headgear frame along the second band.

In at least one embodiment of the airway interface mask and headgear assembly, the assembly also includes a dome cap disposed upon the first tension adjustment knob to aid in the prevention of unwanted head rolls while the patent is sleeping thereby to keep the patient's head facing one direction and not on the backside.

In at least one embodiment of the airway interface mask and headgear assembly, the assembly further includes a cushion disposed upon the air insulation cuff an a side most proximate to the patient's face to provide additional comfort and conformity to the shape and contours of the patient's face particularly over the bridge of the nose of the patient.

In one exemplary embodiment, the technology described herein provides a Continuous Positive Airway Pressure (CPAP) system. The CPAP system includes: an air pump CPAP machine utilized to treat breathing problems such as sleep apnea utilizing mild air pressure to keep a user's airways open; airway interface mask having a mask shell housing to cover the patient's at least one airway interface, such as a mouth, nose, or both; an air hose fluidly coupling the air pump CPAP machine to the airway interface mask; and a pillowed, non-collapsible air cushion consisting of a continuously sealed, generally circular, and hollow cylinder which is limited in compression and which is disposed upon the mask shell housing sealingly in a generally circular manner on an underside of the mask housing to engage the patient's face. The pillowed, non-collapsible air cushion provides the sealed, cushioned engagement to the patient's face necessary, with conformity to the shape and contours of the patient's face, in the CPAP system without allowing for compression of the mask shell housing to allow the mask housing to touch the patient's face.

In at least one embodiment of the CPAP system, the CPAP system also includes: an elbow partition fluidly coupled between the airway interface mask and the air hose; a partition disposed with the elbow partition to provide alternate air flow routes for inhaled air and exhaled air; and a plurality of apertures disposed upon the elbow partition above the partition for exhaled air to exit.

In at least one embodiment of the CPAP system, the CPAP system further includes: an outer ring, disposed around a perimeter of the mask shell housing most proximate to the patient's face when worn, to provide structural support the mask shell housing and with which to couple to the pillowed, non-collapsible air cushion; an adapter ring, disposed around a perimeter of the mask shell housing most distal to the patient's face when worn, to provide structural support the mask shell housing and further comprising a fluid inlet/outlet for air passage with which to couple to an air hose of a CPAP system and provide airflow to the patient through the mask shell housing to the CPAP system; a plurality of headgear connection points; and a protrusion ring, disposed around a perimeter of the adapter ring, to provide the plurality of headgear connection points.

In at least one embodiment of the CPAP system, the CPAP system also includes: a harness with which to couple the airway interface mask to the patient's face; a plurality of straps disposed about the harness; a plurality of apertures disposed about each of the plurality of straps to provide a plurality of connectivity points to the patient to provide multi-point adjustability and to adjust for tightness and comfort; and a plurality of reinforcement lips disposed about at least one side of the plurality of straps at the circumference of each of the plurality of apertures.

In at least one embodiment of the CPAP system, the CPAP system further includes an air insulation cuff comprised of non-porous foam, disposed upon the pillowed, non-collapsible air cushion on a side most proximate to placement upon the patient's face, to provide additional comfort and conformity to the shape and contours of the patient's face.

In at least one embodiment of the CPAP system, the CPAP system also includes: a headgear frame, with which to couple the airway interface mask to the patient's head, the headgear frame comprising a first band for placement about the top portion of the patient's head, from first ear to back of head, to second ear, to forehead, and back to the first ear; and a first tension adjustment knob disposed upon the first band at the back of the head position to tighten or loosen the tension within the headgear frame along the first band.

In at least one embodiment of the CPAP system, the CPAP system further includes a dome cap disposed upon the first tension adjustment knob to aid in the prevention of unwanted head rolls while the patent is sleeping thereby to keep the patient's head facing one direction and not on the backside.

In one exemplary embodiment, the technology described herein provides a method for treating breathing problems such as sleep apnea utilizing mild air pressure to keep a user's airways open. The method includes: utilizing an air pump CPAP machine utilized to treat breathing problems such as sleep apnea utilizing mild air pressure to keep a user's airways open; utilizing an airway interface mask and headgear assembly including: a mask shell housing to cover the patient's at least one airway interface, such as a mouth, nose, or both; and a pillowed, non-collapsible air cushion consisting of a continuously sealed, generally circular, and hollow cylinder which is limited in compression and which is disposed upon the mask shell housing sealingly in a generally circular manner on an underside of the mask housing to engage the patient's face; wherein the pillowed, non-collapsible air cushion provides the sealed, cushioned engagement to the patient's face necessary, with conformity to the shape and contours of the patient's face, in the CPAP system without allowing for compression of the mask shell housing to allow the mask housing to touch the patient's face; placing the airway interface mask over the mouth and nose; moving the airway interface mask for proper placement to ensure no air leaks and to distribute the pressure of the air pillow on the face of the wearer; and actuating the air pump CPAP machine.

In at least one embodiment of the method, the method also includes: utilizing airway interface mask and headgear assembly further including: a plurality of headgear connection points; a protrusion ring, disposed around a perimeter of the adapter ring, to provide the plurality of headgear connection points; a harness with which to couple the airway interface mask to the patient's face; a plurality of straps disposed about the harness; a plurality of apertures disposed about each of the plurality of straps to provide a plurality of connectivity points to the patient to provide multi-point adjustability and to adjust for tightness and comfort; and a plurality of reinforcement lips disposed about at least one side of the plurality of straps at the circumference of each of the plurality of apertures; an air insulation cuff comprised of non-porous foam, disposed upon the pillowed, non-collapsible air cushion on a side most proximate to placement upon the patient's face, to provide additional comfort and conformity to the shape and contours of the patient's face; a headgear frame, with which to couple the airway interface mask to the patient's head, the headgear frame comprising a first band for placement about the top portion of the patient's head, from first ear to back of head, to second ear, to forehead, and back to the first ear; a first tension adjustment knob disposed upon the first band at the back of the head position to tighten or loosen the tension within the headgear frame along the first band; a second band disposed upon the headgear frame for placement about the top portion of the patient's head, from first ear to second ear over the top most portion of the head; a second tension adjustment knob disposed upon the second band to tighten or loosen the tension within the headgear frame along the second band; and a dome cap disposed upon the first tension adjustment knob to aid in the prevention of unwanted head rolls while the patient is sleeping thereby to keep the patient's head facing one direction and not on the backside; and securing the airway interface mask and headgear assembly to the wearer with the harness and headgear frame.

It will be apparent to those skilled in the art, after reading this disclosure, which methods steps disclosed can be performed simultaneously or in a different order than that depicted, or omitted given the nature of a particular procedure.

There has thus been outlined, rather broadly, the more important features of the technology in order that the detailed description thereof that follows may be better understood, and in order that the present contribution to the art may be better appreciated. There are additional features of the technology that will be described hereinafter and which will form the subject matter of the claims appended hereto. In this respect, before explaining at least one embodiment of the technology in detail, it is to be understood that the invention is not limited in its application to the details of construction and to the arrangements of the components set forth in the following description or illustrated in the drawings. The technology described herein is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the technology described herein.

Further objects and advantages of the technology described herein will be apparent from the following detailed description of a presently preferred embodiment which is illustrated schematically in the accompanying drawings.

### BRIEF DESCRIPTION OF THE DRAWINGS

The technology described herein is illustrated with reference to the various drawings, in which like reference numbers denote like device components and/or method steps, respectively, and in which:

FIG. 1 is a perspective view of a CPAP system, illustrating, in particular, a mask assembly, harness, elbow partition, CPAP machine, and an air hose, according to an embodiment of the technology described herein;

FIG. 2 is a front perspective view of the mask assembly depicted in FIG. 1, illustrating, in particular, an air inlet/outlet, an adapter ring, a protrusion ring having a multiplicity of protrusions with which to couple with the harness, the mask shell, outer ring, and air pillow, according to an embodiment of the technology described herein;

FIG. 3 is a top view of the mask assembly depicted in FIG. 2;

FIG. 4 is a top view of the harness depicted in FIG. 1, illustrating, in particular, a base, a multiplicity of connectivity straps, a multiplicity of connectivity holes, with which to couple to the protrusions of the mask assembly, and an expansion zone, according to an embodiment of the technology described herein;

FIG. 5 is a close-up view of the harness depicted in FIGS. 1 and 4, illustrating, in particular, opposing sides of the straps, and the multiplicity of connectivity holes, with which to couple to the protrusions of the mask assembly, and the lip on each hole on one side of the strap, according to an embodiment of the technology described herein;

FIG. 6 is a close-up view of the harness depicted in FIGS. 1, 4, and 5, illustrating, in particular, the lip surrounding each hole, according to an embodiment of the technology described herein;

FIG. 7A is a front perspective view of an elbow partition, illustrating, in particular, an air inlet, an air outlet, and partition, according to an embodiment of the technology described herein;

FIG. 7B is a rear perspective view of an elbow partition, illustrating, in particular, an air inlet, an air outlet, and partition, according to an embodiment of the technology described herein;

FIG. 8 is a perspective view of a switch assembly, illustrating, in particular, a switch, and electrical prongs for insertion into an outlet, according to an embodiment of the technology described herein;

FIG. 9 is a perspective view of an oxygen port fluidly coupled to air hose of the CPAP system, illustrating, in particular, the oxygen port in an open position, according to an embodiment of the technology described herein;

FIG. 10 is a perspective view of an oxygen port fluidly coupled to air hose of the CPAP system, illustrating, in particular, the oxygen port in a closed position, according to an embodiment of the technology described herein;

FIG. 11 is a front perspective view of the mask assembly depicted in FIG. 1, illustrating, in particular, an air inlet/outlet, an adapter ring, a protrusion ring having a multiplicity of protrusions with which to couple with the harness, the mask shell, outer ring, an air pillow, and, in addition to FIG. 2, a foam cuff, according to an embodiment of the technology described herein;

FIG. 12 is a perspective view of a headgear frame, with which to couple the airway interface mask to the patient's head, illustrating, in particular, tension bands for placement about the patient's head, and tension adjustment knobs on the bands and with which to tighten or loosen the tension within the headgear frame, according to an embodiment of the technology described herein;

FIG. 13 is a perspective view of a dome cap disposed upon the first tension adjustment knob to aid in the prevention of unwanted head rolls while the patent is sleeping, thereby to keep the patient's head facing one direction and not on the backside, according to an embodiment of the technology described herein; and

FIG. 14 is a perspective view of a cushion disposed upon the air insulation cuff an a side most proximate to the patient's face to provide additional comfort and conformity to the shape and contours of the patient's face particularly over the bridge of the nose of the patient, according to an embodiment of the technology described herein.

### DETAILED DESCRIPTION OF THE INVENTION

Before describing the disclosed embodiments of this technology in detail, it is to be understood that the technology is not limited in its application to the details of the particular arrangement shown here since the technology described is capable of other embodiments. Also, the terminology used herein is for the purpose of description and not of limitation.

In various exemplary embodiments, the technology described herein provides an apparatus and associated methods for a Continuous Positive Airway Pressure (CPAP) system, which can be utilized to treat breathing problems such as sleep apnea, and the like.

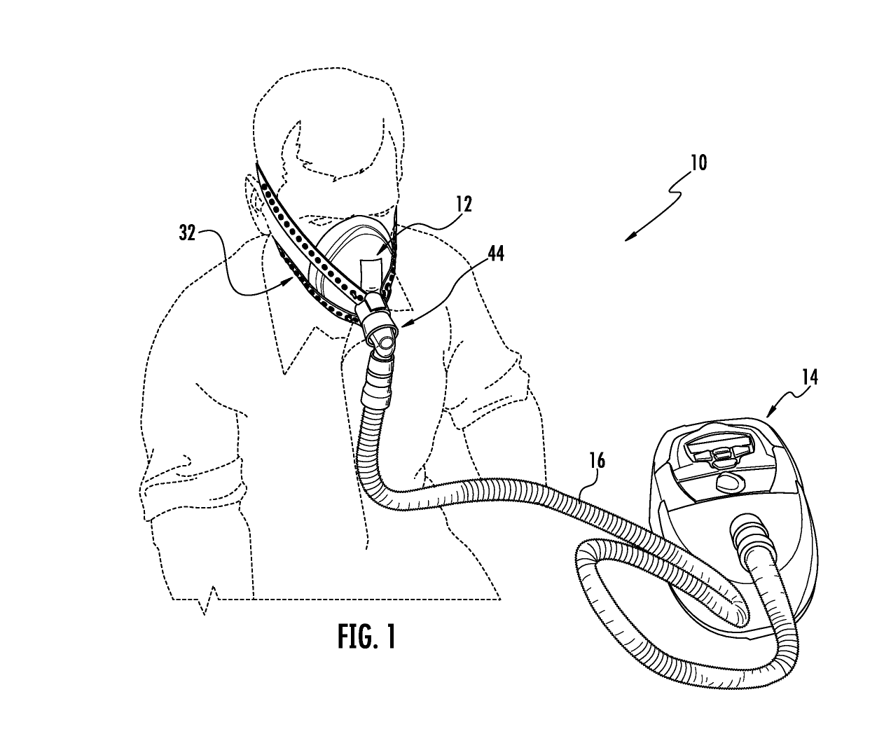

Referring now to FIG. 1, a CPAP system 10 is shown. In general, the CPAP system 10 includes a CPAP machine 14 (having an air pump), a flexible air hose assembly 16, a mask assembly 12, an elbow partition 44, and a harness 32, each of which is described in greater detail in the disclosure below. The CPAP system 10 is shown in use on a wearer with the mask assembly 12 secured over the nose and mouth of the wearer and held in place by a harness 32. The CPAP system 10 is designed to provide high levels of comfort to the wearer.

The CPAP system provides treatment utilizing mild air pressure to keep a user's airways open. CPAP systems are typically used to aid persons with breathing problems such as sleep apnea.

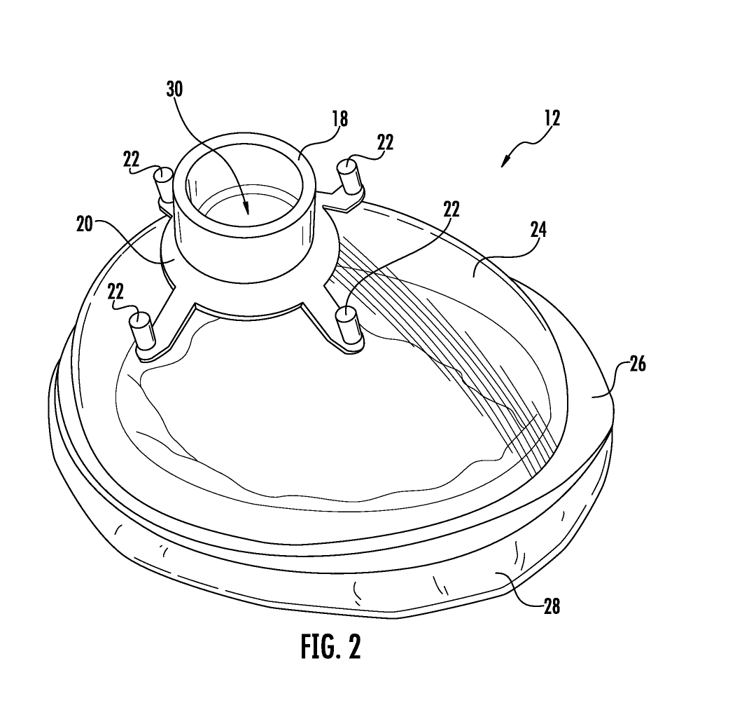

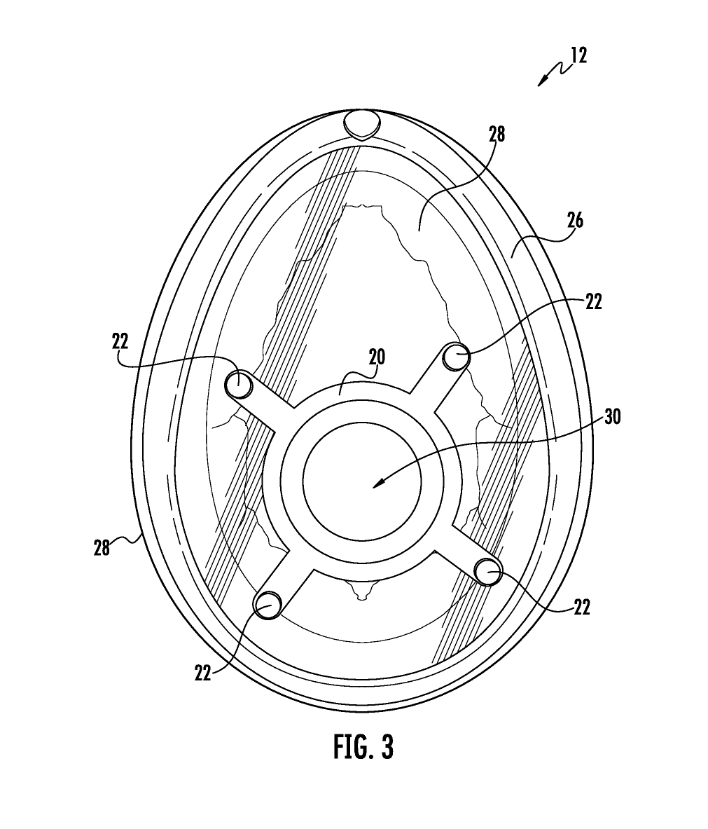

Referring now to FIGS. 2 and 3, the mask assembly 12 of the CPAP system 10 is depicted in greater detail. The mask assembly includes a mask shell 24. The mask shell is typically a firm plastic member being generally a half sphere shape formed for a comfortable fit over both the nose and mouth of the wearer. The mask shell 24 is generally translucent, such that the nose and mouth of the wearer can still be viewed. The mask shell 24 is coupled at one end to an outer ring 26 and coupled at the opposing end to an adapter ring 18.

The mask assembly 12 of the CPAP system 10, in at least one embodiment, is configured for use for one month in duration. By way of example, a six-month supply of the mask assemblies 12 can be retailed to a user. Replacement after a one-month period is utilized for purposes of hygiene and sanitation. Additionally, the mask assembly 12 is configured for use without hook and loop fasteners and without clasps, and the like, which are very difficult to fit precisely.

In at least one embodiment, the mask assembly 12 of the CPAP system 10 is manufactured in 100% non-latex. However, as will be apparent to one of ordinary skill in the art, the mask assembly can be manufactured in alternative materials.

The mask assembly 12 of the CPAP system 10 can be varied in size to accommodate the size of varied users. By way of example, the mask assembly 12 of the CPAP system 10 can be configured in a small, medium, and large size.

The outer ring 26 of mask assembly 12 to which the mask shell 24 is coupled, is further coupled to an air pillow 28. The air pillow 28 is configured to provide a tight and snug, yet comfortable, fit over both the nose and mouth of the wearer and to prevent air leaks. The air pillow 28 is configured to provide a distributed pressure over a contact area. The air pillow 28 provides aid in the prevention air leaks and a comfort fit even as the mask assembly 12 is moved about generally over the nose and mouth of the user.

The adapter ring 18 is configured for coupled to the elbow partition 44. The adapter ring 18 includes a fluid inlet/outlet 30 for air passage. The adapter ring 18 can be fluidly coupled to the elbow partition 44 in a secure, yet interchangeable, fit such that there is no air leak.

The adapter ring 18 is further configured to receive a protrusion ring 20. The protrusion ring 20 slips securely over the outer perimeter of the adapter ring 18. The protrusion ring 20 includes a multiplicity of protrusions 22, or prongs, upon which the harness is attached to securely hold the mask assembly 12 to the wearer.

The adapter ring 18 can be coupled with a swivel. Use of a swivel allows the air hose to rotate without twisting and without placing pressure on the mask assembly while being worn.

In a preferred embodiment, the mask assembly 12 is a single patient use face mask. Such single patient use aids in hygiene and sanitation.

In a preferred embodiment, the mask assembly 12 is disposable. A user can replace the mask assembly 12 on regularly scheduled intervals for the purposes of hygiene and sanitation.

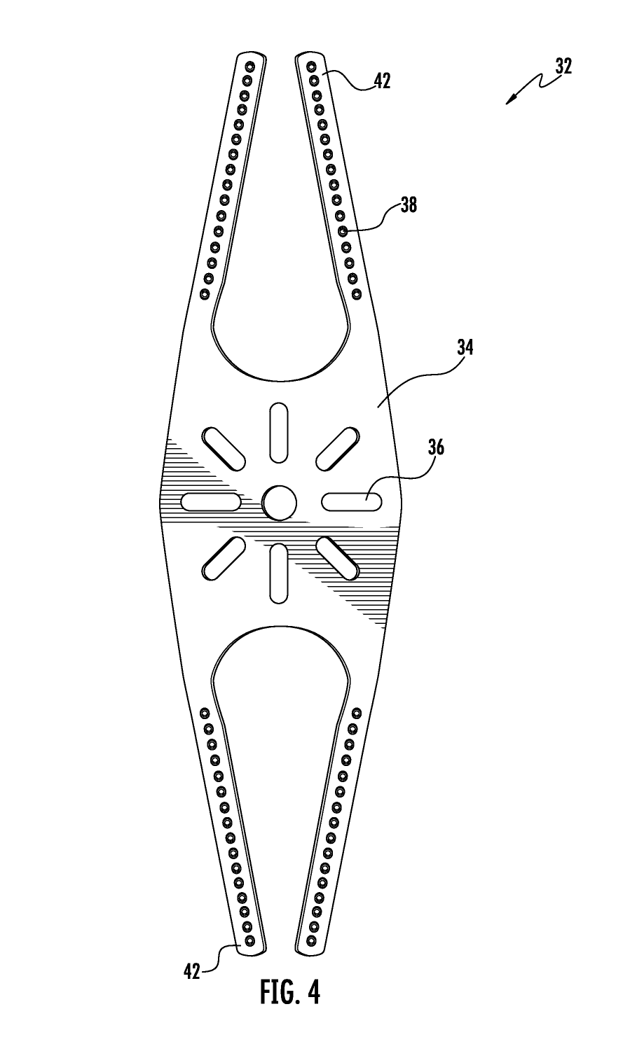

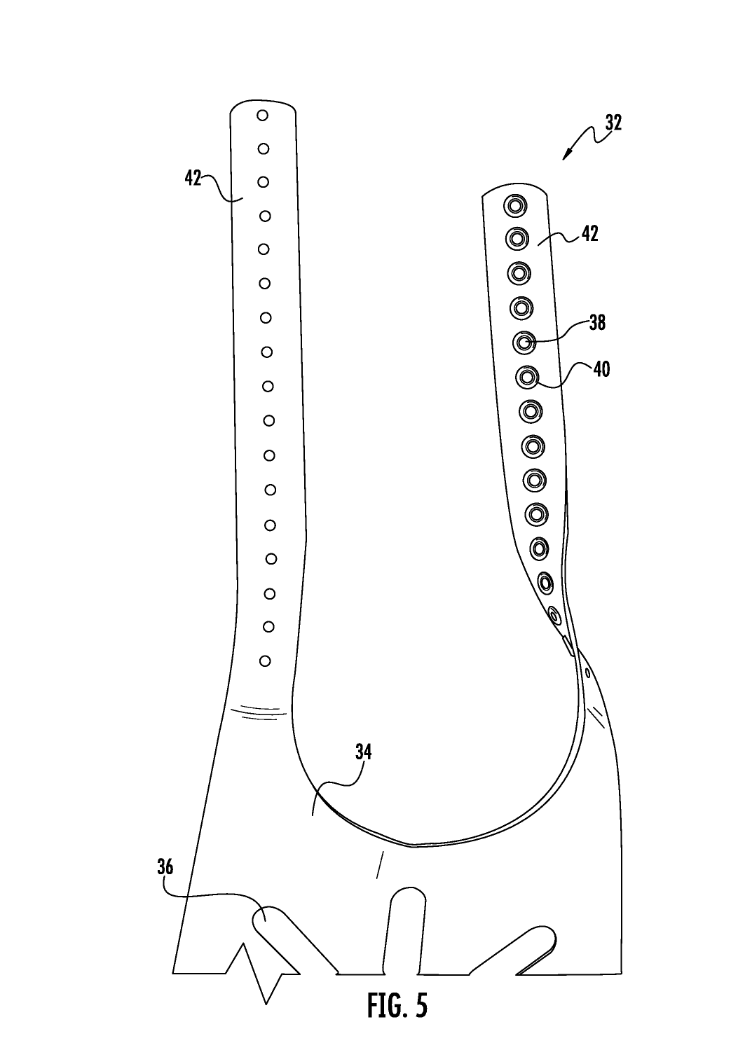

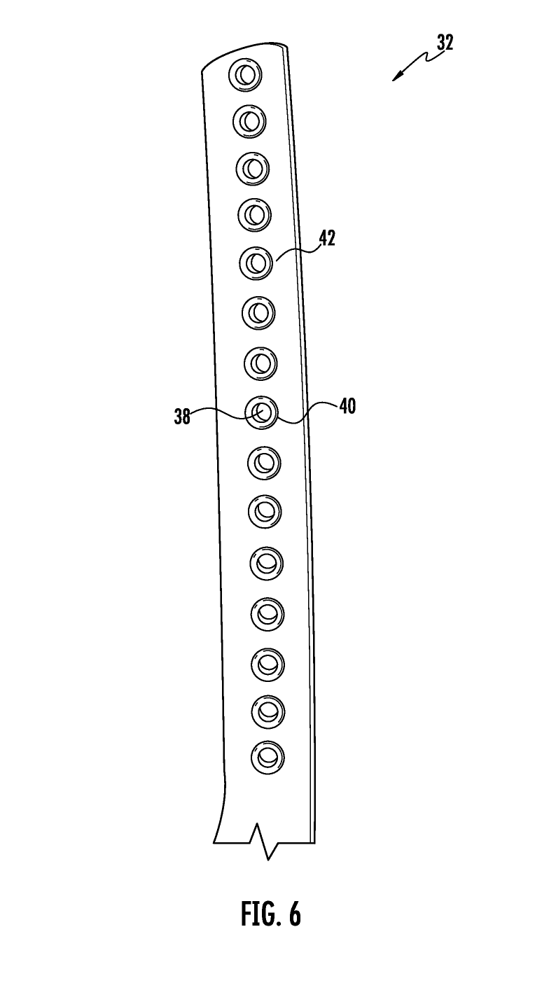

Referring now to FIGS. 4, 5, and 6, the harness 32 is depicted in greater detail. The harness 32 is configured to couple to the mask assembly 12 to securely hold the mask assembly 12 to the wearer. The harness 32 includes a base 34 configured for placement on the back side of the head of the wearer. The base 34 includes an expansion zone 36 such that the harness 32 in that area is expandable to provide comfort and to accommodate varied head sizes.

The harness 32 includes a multiplicity of straps 42. As depicted in FIG. 4, specifically, the harness 32 includes 4 straps; however, the number of straps can be varied as necessitated in specific applications. Each strap 42 includes a multiplicity of holes 38. On one side of each strap 42, each hole includes a reinforcement lip 40.

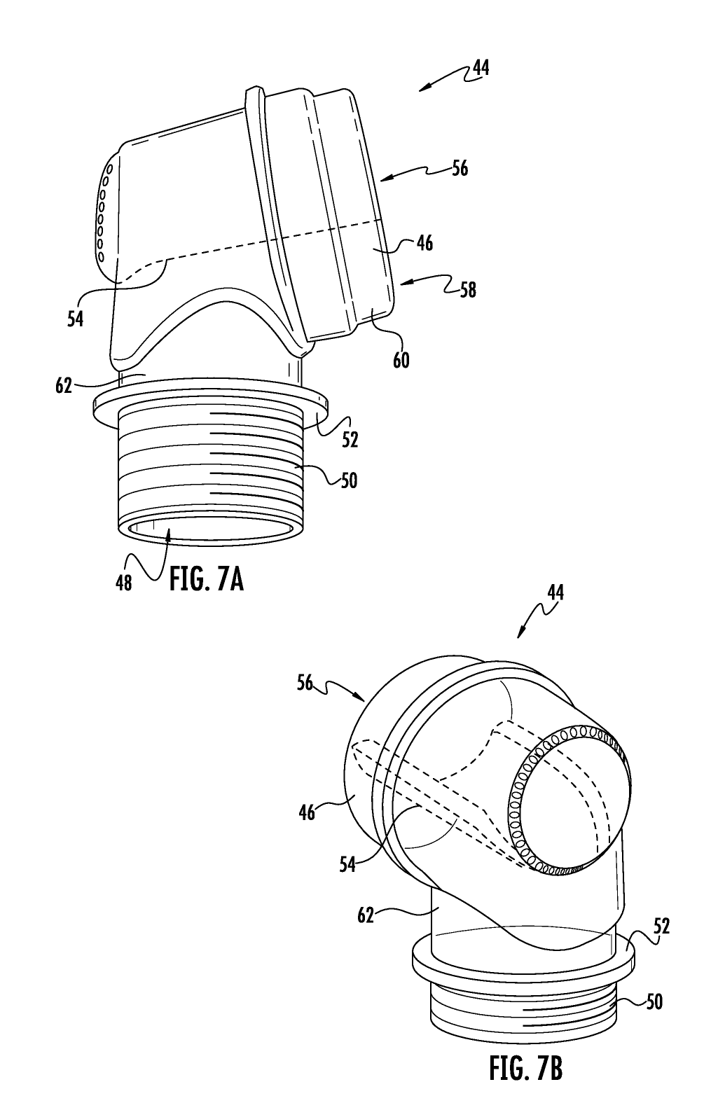

Referring now to FIGS. 7A and 7B, the elbow partition 44 is depicted in greater detail. Air can be drawn in through inlet 48 from the CPAP machine 14 through air hose 16. The elbow partition 44 can be threaded, or otherwise attached, with air hose 16 on threads 50 up to lip 52. Air enters into base 62 and into member 60 for exit through passageway 58. At member 46 air enter into the mask assembly 12. The elbow partition 44 includes partition 54. The partition 54 provides alternative air routes. After air has entered into the mask assembly 12 and is processed by the wearer, exhaled air enters area 56 for exit above partition 54 and through the outlet.

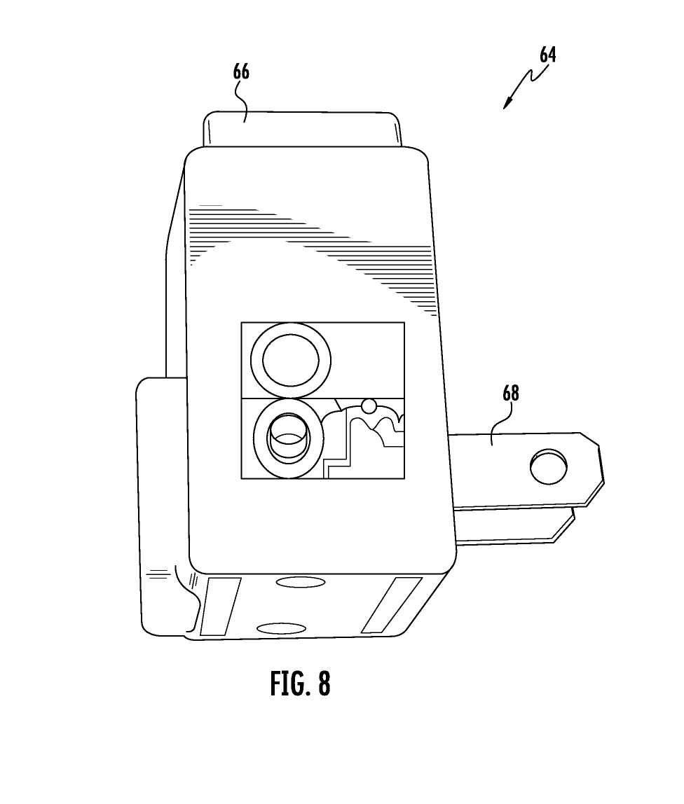

Referring now to FIG. 8, a switch assembly 64 is shown. The switch assembly 68 includes prongs 68 for insertion into a standard electrical outlet. The switch assembly further includes a toggle switch 66, or the like, to activate or deactivate the circuit. The switch assembly 64 is configured for use between an electrical outlet and a CPAP machine 14 air pump. The switch assembly 64 is provided to add additional readiness to the CPAP machine 14 to avoid delays to the user. By way of example, the switch assembly 64 is configured to simplify the control of electricity to the CPAP machine 14.

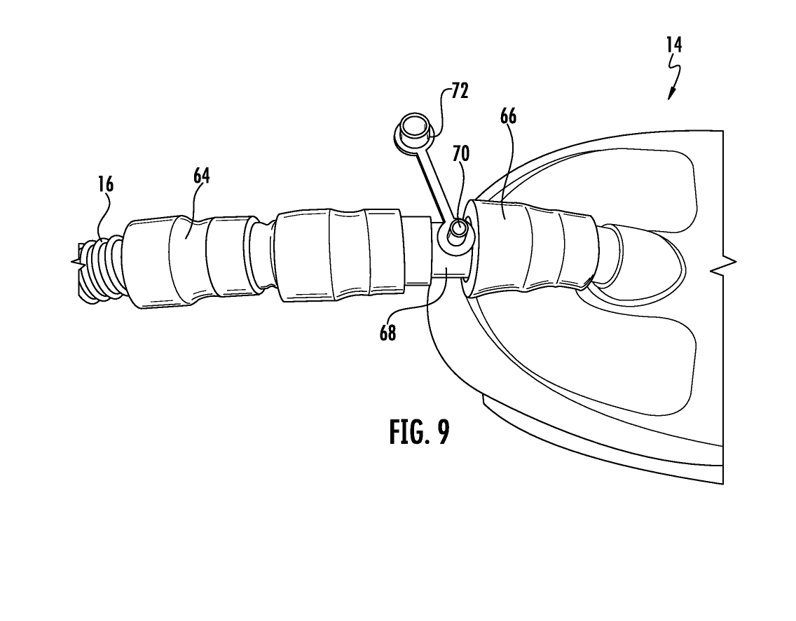

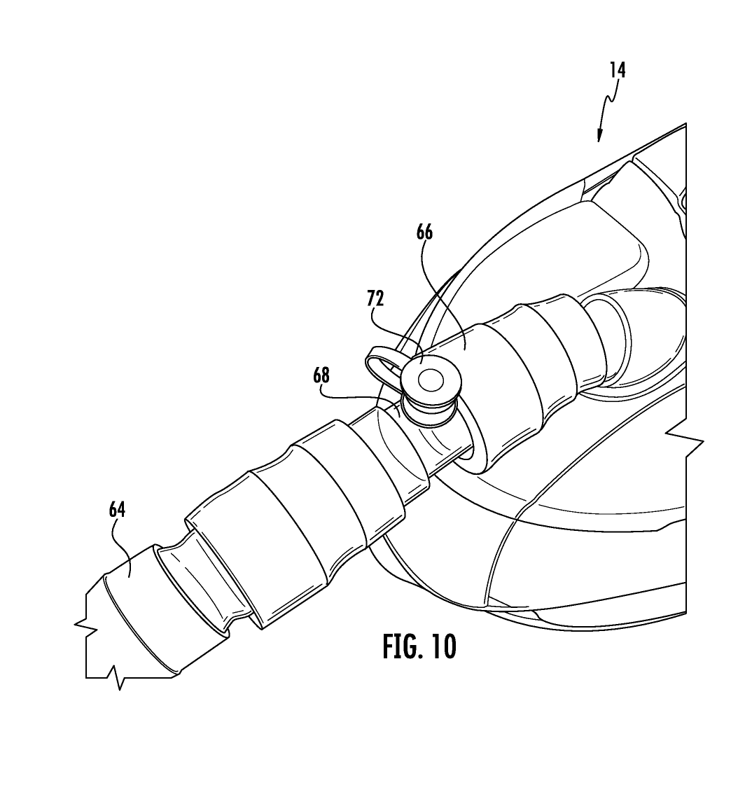

Referring now to FIGS. 9 and 10, an oxygen port 74 is depicted for use with the CPAP machine 14. The oxygen port 74 is configured to be fluidly coupled to the air hose 16 between members 64 and 76. The oxygen port 74 includes input port 70. Oxygen can be coupled to the air hose 16 at oxygen port 74 and enter through input port 70. When the oxygen port 74 is not in use, the input port 70 can be capped with cap 72.

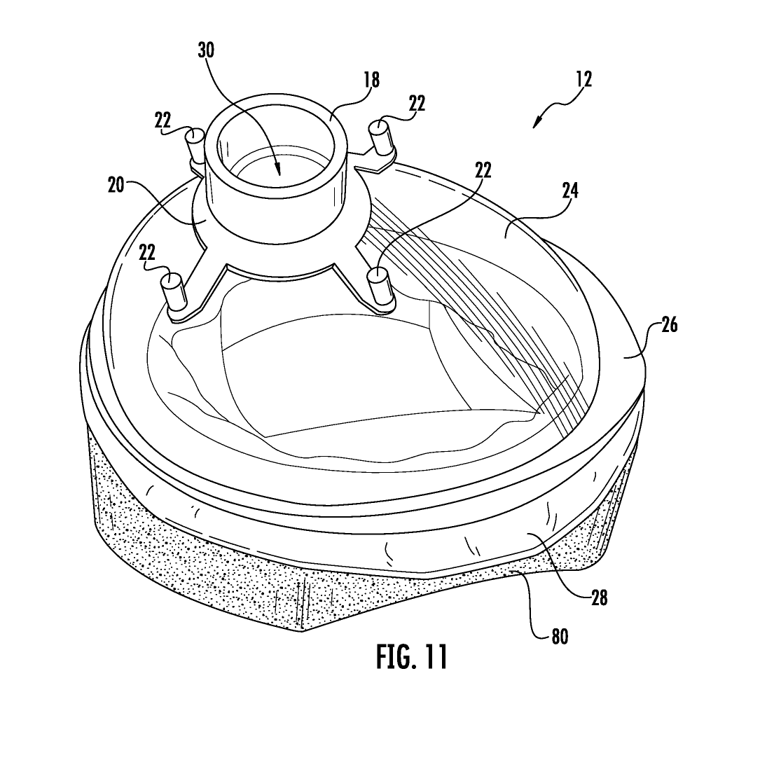

Referring now to FIG. 11, the mask assembly 12 of the CPAP system 10 is depicted in greater detail and in this embodiment, further including an air insulation cuff 80. The air insulation cuff 80 is comprised of non-porous foam in at least one embodiment. The air insulation cuff 80 is disposed upon the pillowed, non-collapsible air cushion 28 on a side most proximate to placement upon the patient's face. The air insulation cuff 80 provides additional comfort and conformity to the shape and contours of the patient's face.

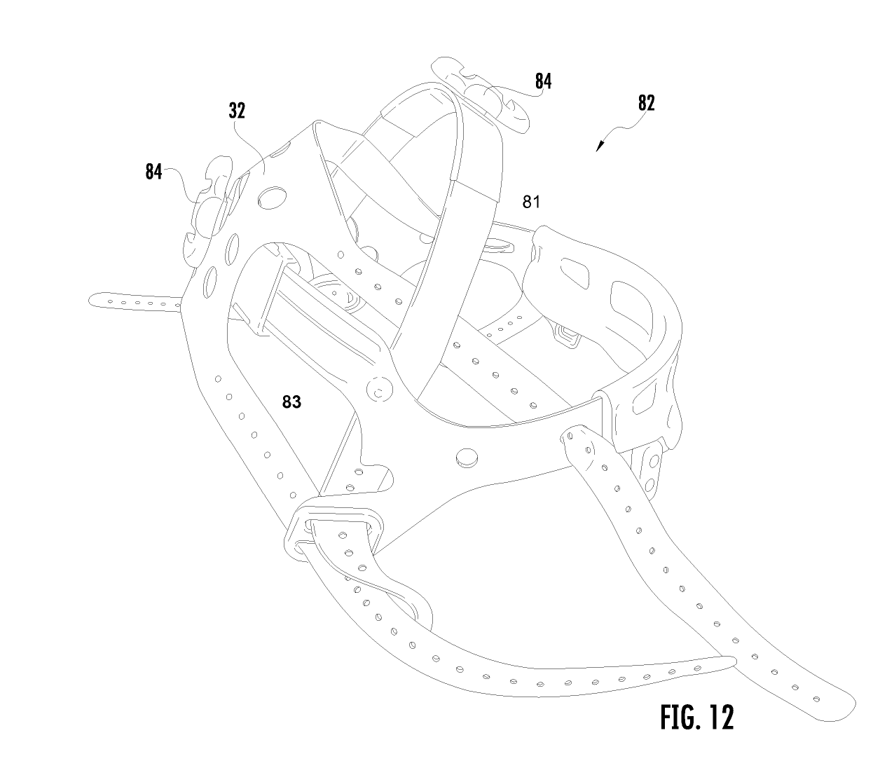

Referring now to FIG. 12, a headgear assembly 82 is depicted. In addition to harness 32 already disclosed, the headgear assembly 82 also includes a headgear frame, with which to couple the airway interface mask to the patient's head. The headgear frame includes a first band 83 for placement about the top portion of the patient's head, from first ear to back of head, to second ear, to forehead, and back to the first ear. The first band 83 includes a first tension adjustment knob 84 disposed upon the first band 83 at the back of the head position to tighten or loosen the tension within the headgear frame along the first band.

In at least one embodiment, the headgear assembly 82 also includes a second band 81 disposed upon the headgear frame for placement about the top portion of the patient's head, from first ear to second ear over the top most portion of the head. The second band 81 includes a second tension adjustment knob 84 disposed upon the second band 81 to tighten or loosen the tension within the headgear frame along the second band.

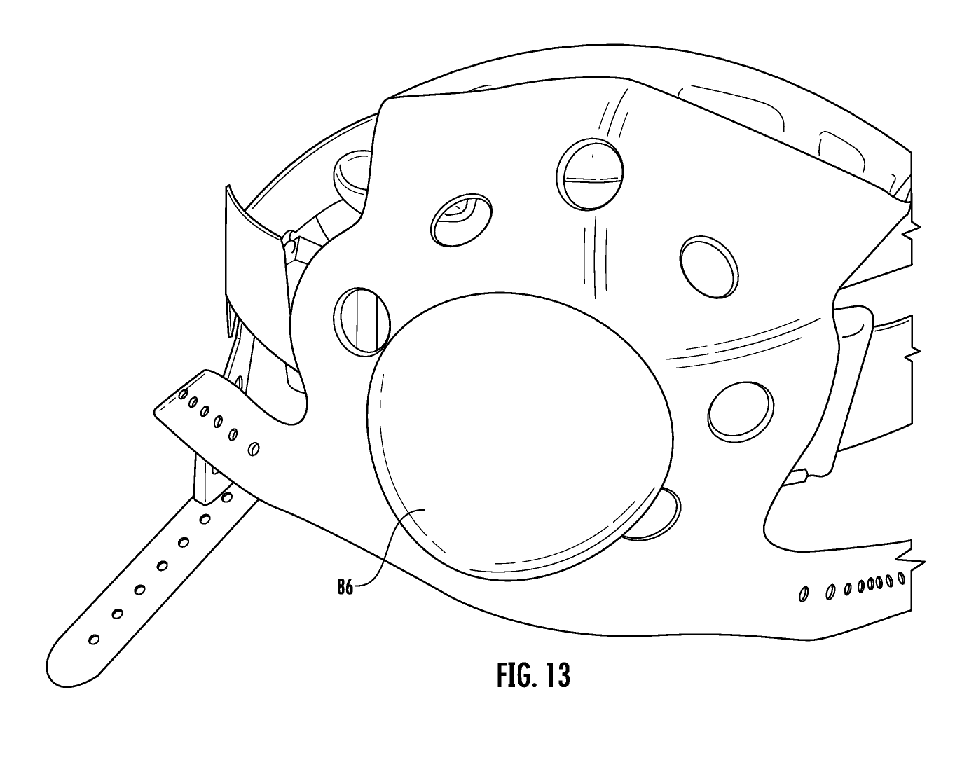

Referring now to FIG. 13, a dome cap 86 is depicted. The dome cap 86 is disposed upon the first tension adjustment knob 84 to aid in the prevention of unwanted head rolls while the patent is sleeping, thereby to keep the patient's head facing one direction and not on the backside. Alternative shapes and sizes are utilized in various embodiments.

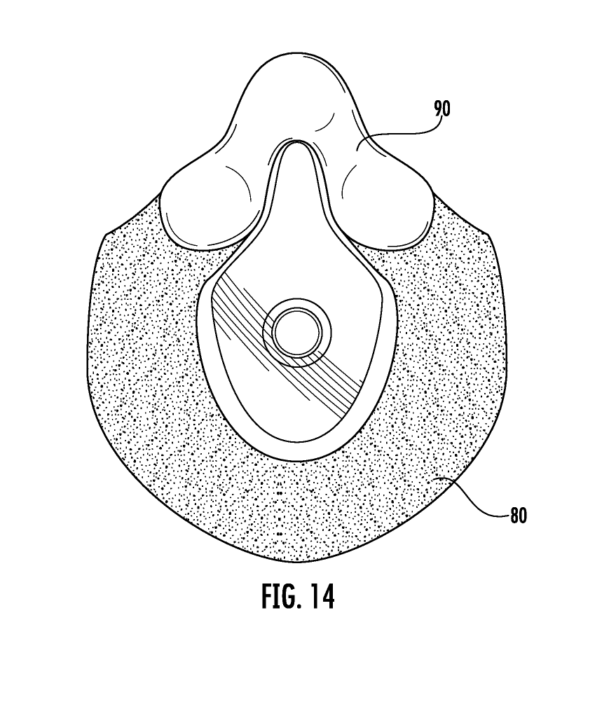

Referring now to FIG. 14, a cushion 90 is depicted. The cushion 90 is disposed upon the air insulation cuff 80 on a side most proximate to the patient's face to provide additional comfort and conformity to the shape and contours of the patient's face particularly over the bridge of the nose of the patient. In at least one embodiment, the cushion 90 is adhered to the air insulation cuff 80 with an adhesive. In at least one embodiment, the cushion 90 is formed of a plastic gel.

In operation, and by way of example, the CPAP system 10, as disclosed and claimed, can be utilized by the following methods steps:
utilizing a mask assembly;
placing the mask assembly over the mouth and nose;
moving the mask assembly for proper placement to ensure no air leaks and to distribute the pressure of the air pillow on the face of the wearer;
utilizing a mask harness;
securing the mask assembly to the wearer with the harness; and
actuating the air pump CPAP machine.

Additional method steps can include:
utilizing a swivel in the air hose at the point of connectivity with the CPAP machine air pump;
utilizing a swivel in the air hose at the point of connectivity with the mask assembly and elbow partition;
utilizing an oxygen port within the air hose to optionally add oxygen into the air flow for the user;

It will be apparent to those skilled in the art, after reading this disclosure, which methods steps disclosed can be performed simultaneously or in a different order than that depicted, or omitted given the nature of a particular procedure.

Although this technology has been illustrated and described herein with reference to preferred embodiments and specific examples thereof, it will be readily apparent to those of ordinary skill in the art that other embodiments and examples can perform similar functions and/or achieve like results. All such equivalent embodiments and examples are within the spirit and scope of the invention and are intended to be covered by the following claims.

What is claimed is:

1. An airway interface mask and headgear assembly for a patient Continuous Positive Airway Pressure (CPAP) system, utilized to treat breathing problems such as sleep apnea utilizing mild air pressure to keep a user's airways open, the airway interface mask and headgear assembly comprising:
a mask shell housing to cover the patient's at least one airway interface; and
a pillowed, non-collapsible air cushion consisting of a continuously sealed, generally circular, and hollow cylinder which is limited in compression and which is disposed upon the mask shell housing sealingly in a generally circular manner on an underside of the mask housing to engage the patient's face;
wherein the pillowed, non-collapsible air cushion provides the sealed, cushioned engagement to the patient's face necessary, with conformity to the shape and contours of the patient's face, in the CPAP system without allowing for compression of the mask shell housing to allow the mask housing to touch the patient's face.

2. The airway interface mask and headgear assembly of claim 1, further comprising:
an outer ring, disposed around a perimeter of the mask shell housing most proximate to the patient's face when worn, to provide structural support the mask shell housing and with which to couple to the pillowed, non-collapsible air cushion.

3. The airway interface mask and headgear assembly of claim 1, further comprising:
an adapter ring, disposed around a perimeter of the mask shell housing most distal to the patient's face when worn, to provide structural support the mask shell housing and further comprising a fluid inlet/outlet for air passage with which to couple to an air hose of a CPAP system and provide airflow to the patient through the mask shell housing to the CPAP system.

4. The airway interface mask and headgear assembly of claim 3, further comprising:
a plurality of headgear connection points; and
a protrusion ring, disposed around a perimeter of the adapter ring, to provide the plurality of headgear connection points.

5. The airway interface mask and headgear assembly of claim 4, further comprising:
a harness with which to couple the airway interface mask to the patient's face;
a plurality of straps disposed about the harness;
a plurality of apertures disposed about each of the plurality of straps to provide a plurality of connectivity points to the patient to provide multi-point adjustability and to adjust for tightness and comfort; and
a plurality of reinforcement lips disposed about at least one side of the plurality of straps at the circumference of each of the plurality of apertures.

6. The airway interface mask and headgear assembly of claim 3, further comprising:
a swivel coupled to the adapter ring to allow an air hose to rotate without twisting and without placing pressure on the mask shell housing while being worn by the patient.

7. The airway interface mask and headgear assembly of claim 1, further comprising:
an air insulation cuff comprised of non-porous foam, disposed upon the pillowed, non-collapsible air cushion on a side most proximate to placement upon the patient's face, to provide additional comfort and conformity to the shape and contours of the patient's face.

8. The airway interface mask and headgear assembly of claim 1, further comprising:
a headgear frame, with which to couple the airway interface mask to the patient's head, the headgear frame comprising a first band for placement about the top portion of the patient's head, from first ear to back of head, to second ear, to forehead, and back to the first ear; and
a first tension adjustment knob disposed upon the first band at the back of the head position to tighten or loosen the tension within the headgear frame along the first band.

9. The airway interface mask and headgear assembly of claim 8, further comprising:
a second band disposed upon the headgear frame for placement about the top portion of the patient's head, from first ear to second ear over the top most portion of the head; and
a second tension adjustment knob disposed upon the second band to tighten or loosen the tension within the headgear frame along the second band.

10. The airway interface mask and headgear assembly of claim 8, further comprising:
a dome cap disposed upon the first tension adjustment knob to aid in the prevention of unwanted head rolls while the patent is sleeping thereby to keep the patient's head facing one direction and not on the backside.

11. The airway interface mask and headgear assembly of claim 8, further comprising:
a cushion disposed upon the air insulation cuff on a side most proximate to the patient's face to provide additional comfort and conformity to the shape and contours of the patient's face particularly over the bridge of the nose of the patient.

12. A Continuous Positive Airway Pressure (CPAP) system comprising:
an air pump CPAP machine utilized to treat breathing problems such as sleep apnea utilizing mild air pressure to keep a user's airways open;
airway interface mask having a mask shell housing to cover the patient's at least one airway interface;
an air hose fluidly coupling the air pump CPAP machine to the airway interface mask; and
a pillowed, non-collapsible air cushion consisting of a continuously sealed, generally circular, and hollow cylinder which is limited in compression and which is disposed upon the mask shell housing sealingly in a generally circular manner on an underside of the mask housing to engage the patient's face;
wherein the pillowed, non-collapsible air cushion provides the sealed, cushioned engagement to the patient's face necessary, with conformity to the shape and contours of the patient's face, in the CPAP system without allowing for compression of the mask shell housing to allow the mask housing to touch the patient's face.

13. The CPAP system of claim 12, further comprising:
an elbow partition fluidly coupled between the airway interface mask and the air hose;
a partition disposed with the elbow partition to provide alternate air flow routes for inhaled air and exhaled air; and
a plurality of apertures disposed upon the elbow partition above the partition for exhaled air to exit.

14. The CPAP system of claim 12, further comprising:
an outer ring, disposed around a perimeter of the mask shell housing most proximate to the patient's face when worn, to provide structural support the mask shell housing and with which to couple to the pillowed, non-collapsible air cushion;
an adapter ring, disposed around a perimeter of the mask shell housing most distal to the patient's face when worn, to provide structural support the mask shell housing and further comprising a fluid inlet/outlet for air passage with which to couple to an air hose of a CPAP system and provide airflow to the patient through the mask shell housing to the CPAP system;
a plurality of headgear connection points; and
a protrusion ring, disposed around a perimeter of the adapter ring, to provide the plurality of headgear connection points.

15. The CPAP system of claim 14, further comprising:
a harness with which to couple the airway interface mask to the patient's face;
a plurality of straps disposed about the harness;
a plurality of apertures disposed about each of the plurality of straps to provide a plurality of connectivity points to the patient to provide multi-point adjustability and to adjust for tightness and comfort; and
a plurality of reinforcement lips disposed about at least one side of the plurality of straps at the circumference of each of the plurality of apertures.

16. The CPAP system of claim 12, further comprising:
an air insulation cuff comprised of non-porous foam, disposed upon the pillowed, non-collapsible air cushion on a side most proximate to placement upon the patient's face, to provide additional comfort and conformity to the shape and contours of the patient's face.

17. The CPAP system of claim 12, further comprising:
a headgear frame, with which to couple the airway interface mask to the patient's head, the headgear frame comprising a first band for placement about the top portion of the patient's head, from first ear to back of head, to second ear, to forehead, and back to the first ear; and
a first tension adjustment knob disposed upon the first band at the back of the head position to tighten or loosen the tension within the headgear frame along the first band.

18. The CPAP system of claim 17, further comprising:
a dome cap disposed upon the first tension adjustment knob to aid in the prevention of unwanted head rolls while the patent is sleeping thereby to keep the patient's head facing one direction and not on the backside.

19. A method for treating breathing problems such as sleep apnea utilizing mild air pressure to keep a user's airways open, the method comprising:
utilizing an air pump CPAP machine utilized to treat breathing problems such as sleep apnea utilizing mild air pressure to keep a user's airways open;
utilizing an airway interface mask and headgear assembly comprising: a mask shell housing to cover the patient's at least one airway interface; and a pillowed, non-collapsible air cushion consisting of a continuously sealed, generally circular, and hollow cylinder which is limited in compression and which is disposed upon the mask shell housing sealingly in a generally circular manner on an underside of the mask housing to engage the patient's face; wherein the pillowed, non-collapsible air cushion provides the sealed, cushioned engagement to the patient's face necessary, with conformity to the shape and contours of the patient's face, in the CPAP system without allowing for compression of the mask shell housing to allow the mask housing to touch the patient's face;
placing the airway interface mask over the mouth and nose;
moving the airway interface mask for proper placement to ensure no air leaks and to distribute the pressure of the air pillow on the face of the wearer; and
actuating the air pump CPAP machine.

20. The method of claim 19, further comprising:
utilizing airway interface mask and headgear assembly further comprising: a plurality of headgear connection points; a protrusion ring, disposed around a perimeter of the adapter ring, to provide the plurality of headgear connection points; a harness with which to couple the airway interface mask to the patient's face; a plurality of straps disposed about the harness; a plurality of apertures disposed about each of the plurality of straps to provide a plurality of connectivity points to the patient to provide multi-point adjustability and to adjust for tightness and comfort; and a plurality of reinforcement lips disposed about at least one side of the plurality of straps at the circumference of each of the plurality of apertures; an air insulation cuff comprised of non-porous foam, disposed upon the pillowed, non-collapsible air cushion on a side most proximate to placement upon the patient's face, to provide additional comfort and conformity to the shape and contours of the patient's face; a headgear frame, with which to couple the airway interface mask to the patient's head, the headgear frame comprising a first band for placement about the top portion of the patient's head, from first ear to back of head, to second ear, to forehead, and back to the first ear; a first tension adjustment knob disposed upon the first band at the back of the head position to tighten or loosen the tension within the headgear frame along the first band; a second band disposed upon the headgear frame for placement about the top portion of the patient's head, from first ear to second ear over the top most portion of the head; a second tension adjustment knob disposed upon the second band to tighten or loosen the tension within the headgear frame along the second band; and a dome cap disposed upon the first tension adjustment knob to aid in the prevention of unwanted head rolls while the patent is sleeping thereby to keep the patient's head facing one direction and not on the backside; and
securing the airway interface mask and headgear assembly to the wearer with the harness and headgear frame.
